(19) United States
(12) Patent Application Publication
(10) Pub. No.: US 2008/0255429 A1
(43) Pub. Date: Oct. 16, 2008

(54) SIMULATED SECONDARY GAIN MODEL WITH CONTINUOUS FEEDBACK TO DIAGNOSTIC TESTING PARAMETERS AND INTERPRETATION IN A MEDICAL LEGAL SETTING

(76) Inventor: Christopher Centeno, Boulder, CO (US)

Correspondence Address: Christopher J. Centeno, M.D., 541 Highland Ave, Boulder, CO, 80302, US

(21) Appl. No.: 11/733,802

(22) Filed: Apr. 11, 2007

(51) Int. Cl. A61B 5/00

(52) U.S. Cl. 600/300

(57) ABSTRACT

The invention generally relates to compositions and methods for the use of a simulated secondary gain model for use in medical legal setting where there is dispute about the validity of a medical patient's injuries. This model is used to ascertain the false positive rate of a given diagnostic test or suite of same and is then used to reevaluate the test on an ongoing basis after changes have been made to reduce the false positive rate.

### TECHNICAL FIELD

This invention generally relates to methods and compositions for the use of false positive information provided by a simulated secondary gain model of diagnostic testing to produce diagnostic tests with low or no false positive rates when used in medical-legal environments.

### BACKGROUND OF THE INVENTION

Medical legal settings can produce uncertainty in patient diagnosis. Many medical authors have commented that worker's compensation claims can increase the cost of medical care due to secondary gain. (Modlin 1986; Sander and Meyers 1986; Frieman and Fenlin 1995; Kaptain, Shaffrey et al. 1999). Several authors have also had the same comments about automobile insurance claims for medical benefits and disability caused by car crashes (Ferrari 2000; Partheni, Constantoyannis et al. 2000; Ferrari and Schrader 2001). While worker's compensation reforms have produced some results in lowered claim costs in this setting, it was the automobile insurance industry who instituted the best documented changes in claims handling policies based on this suspected phenomenon. In general, this aggressive claims stance was fueled by data from the RAND Corporation that estimates that approximately 42% of reported “soft-injury” claims are for nonexistent or preexisting injuries (American Law and Economics Review V3 N2 2001 (228-250)). As a result of earlier RAND and other studies, the U.S. automobile insurance industry launched a new concept in claims handling called M.I.S.T., an acronym for Minor Impact Soft Tissue ((Centeno, Freeman et al. 2005)). The theory behind this claims stance was that it was virtually impossible to sustain a permanent or serious injury in a low damage car crash. In addition, any claims without objective medical evidence (often in the form of diagnostic imaging) to support complaints and reported disability, would be placed into a “soft-tissue” category. The attitude of insurers is that these “soft-tissue” claims should be handled differently. This claims handling policy has expanded to almost all major U.S. insurers, yet little has been published regarding its scientific validity. For many patients with objective physical exam findings but little automobile property damage, this policy has led to loss of insurance coverage for their injuries. The same has held true for patients with little objective evidence of injury regardless of property damage. Worker's compensation has a similar landscape for injuries without objective evidence on diagnostic testing. For injuries in this “soft-tissue” category and other claims the National Insurance Crime Bureau in Palos Hills, Ill. states that workers' compensation fraud costs the insurance industry $5 billion every year.

Despite studies by the insurance industry that fabricated injury and the resultant build-up of insurance claims cost society billions of dollars, the research supporting that many of these same patients have very real physical injuries has increased with new research into the basis for chronic pain. Freeman was the first to point out that many of the studies refuting the existence of ongoing long-term disability and symptoms in patients diagnosed with whiplash had very poor methodology. In addition, medical research into the injury mechanisms and pathophysiology of whiplash injuries, soft-tissue injuries, and lumbar injuries has exploded in recent years. Much of this has been fueled by a clearer understanding of what causes chronic pain. As a result of this new scientific information, the landscape has been significantly altered. What was called a “soft-tissue” injury has now been redefined into numerous injury diagnosis categories.

The research into whiplash is illustrative of this new scientific knowledge. Seminal studies by Taylor and Twomey demonstrated that serious spinal injuries from car crashes could be detected on cadaver dissection when no imaging evidence of an injury could be found. (Twomey, Taylor et al. 1989; Taylor and Twomey 1993; Taylor, Twomey et al. 1998) This patient cohort had all died of other causes such as blunt abdominal trauma, yet many seemed to have very serious spinal injuries. These injuries included bleeding into the dorsal root ganglia, small fractures of the facet joints, bleeding into the facet joints, and other injuries. Again, while these insults could be easily detected on dissection, they couldn't be detected on more advanced imaging.

In-vitro studies by Grauer and Panjabi are also telling. In simulated low speed rear end collisions, they demonstrated facet joint spearing in the cervical spine as well as significant ligament stretch injury to the anterior longitudinal ligament and facet joint capsules. (Grauer, Panjabi et al. 1997; Panjabi, Cholewicki et al. 1998; Panjabi, Cholewicki et al. 1998) Other authors have now confirmed these findings and added to the database of significant joint and ligament injuries that occur at low speeds. (Yoganandan, Pintar et al. 1998; Yoganandan, Pintar et al. 2002) In addition, these findings have also been confirmed in live volunteers in simulated low speed crash tests. (Kaneoka, Ono et al. 1999)

More recently, neurologic causes of pain have been the focus of research into why some whiplash patients continue to report symptoms long after a muscle strain or ligament sprain should have healed. The early studies above demonstrating injury to the dorsal root ganglion as well as crash research by Svensson showing injury to the same structure, has moved researchers to take a closer look at neurologic injury as a cause of what would be termed by the insurance industry as “soft-tissue injury”. (Ortengren, Hansson et al. 1996; Svensson, Aldman et al. 1998; Eichberger, Darok et al. 2000; Svensson, Bostrom et al. 2000) It has been noted by numerous researchers that late whiplash patients have different sensory thresholds than normal controls. (Curatolo, Petersen-Felix et al. 2001; Moog, Quintner et al. 2002; Sterling, Treleaven et al. 2002; Sterling, Jull et al. 2003; Sterling 2004; Sterling, Jull et al. 2004) These patients show increased sensitivity to a variety of stimuli including pressure, light vibration, heat and cold, not only in the neck but also in body areas remote to the site of pain such as the front of the shin. This means that they feel things differently than someone with a normal sensory system. Importantly in those patients who fail to recover following injury, these sensory changes have been shown to be present from very soon after injury.

Finally, investigators over the last decade have reported that serious ligament injury is likely one cause of some late whiplash complaints. Despite being frequently labeled in a “soft-tissue” injury category, specialized MRI's in these patients show indicators of ligament injuries in the alar, transverse ligament, posterior atlanto-occipital membrane and tectorial membranes. These same injury patterns are not seen in normal controls. (Krakenes, Kaale et al. 2002; Krakenes, Kaale et al. 2003; Krakenes, Kaale et al. 2003; Krakenes, Kaale et al. 2004) In addition, significant lower cervical ligament injury has also been reported by multiple authors both in vitro cadaver studies and in real world imaging studies. (Dvorak, Panjabi et al. 1993; Griffiths, Olson et al. 1995; Yoganandan, Cusick et al. 2001; Stemper, Yoganandan et al. 2002; Yoganandan, Pintar et al. 2002; Kristjansson, Leivseth et al. 2003; Stemper, Yoganandan et al. 2003)

The spinal injury situation associated with worker's compensation injuries to the low back is similar. For example, muscle atrophy in this same soft tissue patient population often accused of malingering has now been detected in the lumbar spine, even when the MRI has been otherwise read as negative. (Hides, Stokes et al. 1994; Hides, Richardson et al. 1995; Hides, Richardson et al. 1996; Solomonow, Zhou et al. 1998; Kader, Wardlaw et al. 2000; Holm, Indahl et al. 2002; Kristjansson 2004) This muscle atrophy has been associated with patient complaints of leg pain, again even in situations where most physicians would read the MRI as a negative study.

Despite this data, there is a significant paucity of good diagnostic tests that are capable of distinguishing injured patients from normal controls. However, even in this area, some progress has been made. Tests such as QST (Quantitative Somatosensory testing)(Sterling, Jull et al. 2003; Sterling 2004; Sterling, Jull et al. 2004), ENG or VNG with Smooth Pursuit Neck torsion testing (Gimse, Tjell et al. 1996; Tjell and Rosenhall 1998), and various proprioceptive evaluations show great promise (Sterling, Jull et al. 2003; Treleaven, Jull et al. 2003; Sterling, Jull et al. 2004). However, the susceptibility of these diagnostic tests to malingering or manipulation is not known.

In essence, there is a mounting dichotomy between the views of insurers and employers who see rampant fraud in situations where a “soft-tissue” injury is diagnosed and the medical science, which increasingly is discovering valid organic etiologies for these complaints and also justifying the severity of their clinical presentation. Clearly, a diagnostic testing methodology is needed to settle these disputes, which frequently clog up the court systems of many countries. This diagnostic testing methodology must take into account the fact that in every medical legal situation, there likely are patients who are trying to defraud the system for monetary gain and actual injured or ill patients where diagnosis is difficult. As a result, any diagnostic testing in this setting must be able to distinguish between these two groups with high accuracy.

### SUMMARY OF THE INVENTION

Embodiments of the invention provide compositions and methods for the use of a simulated secondary gain model in a medical legal setting where there is dispute about the validity of a patient's injuries or illness. This model is used to ascertain the false positive rate of a given diagnostic test by performing said test on a population of subjects without disease who have been incentivized to feign illness and then comparing the rates of abnormal test results to a population of patients with known disease and/or a population without known disease. The false positive data produced by this simulated secondary gain trial is then used to alter the test parameters or interpretation to reduce the false positive rate of patients with real secondary gain on subsequent tests.

### BRIEF DESCRIPTION OF THE DRAWINGS

FIG. 1 demonstrates the feedback loop described herein in a graphical format.

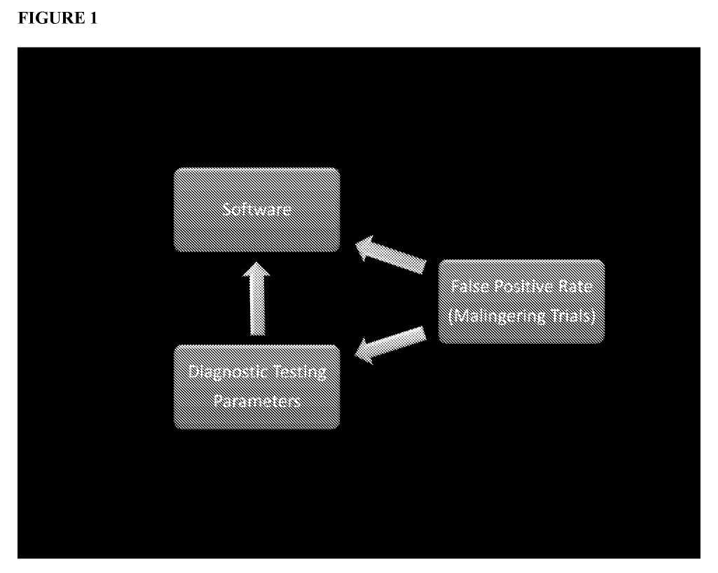

### DETAILED DESCRIPTION OF THE INVENTION

Aspects of the invention include a “Simulated Secondary Gain Model”. This model involves a “target” test or tests to be verified or improved for false positive errors, a group of subjects who have been screened not to have the disease being detected by the diagnostic test, a group of patients known to have the disease being detected, a secondary gain “prize” (provided to the subject or subjects most able to produce false positive results on the test or tests being evaluated) which is of significant value to the subjects such that they will give their best effort, and a group of test readers who are blinded to the identity of the test subjects.

Aspects of the invention also include that the false positive rate, true positive rate, false negative rate, true negative rate, sensitivity, specificity, positive predictive value, and negative predictive value are calculated for the target test using the above methodology. This is produced by having the “blinded readers” (who are blind to which subjects actually have the disease and which are in a simulated secondary gain environment) score each test as “normal” or abnormal”.

This invention uses the following standard definitions:

| | | S+ | S− | Total |
|---|---|---|---|---|
| Clinical | T+ | a = True | b = False | a + b |
| Test | | Positive | Positive | |
| | T− | c = False | d = True | c + d |
| | | Negative | Negative | |
| | Total | a + c | b + d | |

Where Sensitivity=a/(a+c), Specificity=d/(b+d), Positive Predictive Value (PPV)=a/(a+b), and Negative Predictive Value (NPV)=d/(c+d).

While the idea of a false negative rate for a diagnostic test is not new, the concept is anchored in the idea of detecting disease, namely that a false positive would result if a diagnostic test indicated the presence of disease in a patient without disease. While the diagnostic tests being evaluated by this invention can detect disease and as such must have high sensitivity and specificity for a disease state, they must also have another property which allows them to be impervious or nearly impervious to conscious deception. For the purposes of this discussion, this property will be called the “Malingering False Positive” rate. So while a diagnostic test may have a very low disease false positive rate allowing it to rarely show positive when the disease is not present, it may have a very poor “Malingering False Positive” rate, making it easy to fake a positive result by a motivated patient.

While this is not meant to limit the scope of this invention, both disease false/true positive/negative rates as well as malingering false/true positive/negative rates are reported for each test evaluated by the invention. These rates form the basis of the feedback loop which allows the diagnostic test to evolve to achieve better accuracy in both a medical and legal setting.

Aspects of the invention also include a simulated secondary gain environment that promotes the fraudulent testing behavior which can be seen in a legal setting with monetary gain. The key idea behind secondary gain as described by the legal, medical, and insurance communities is that a patient stands to gain attention, money, or both from successfully convincing others (including his health care providers, family, and friends) that he or she is sick (when no or minimal illness is actually present). This culminates in the court room or legal setting where there is an insurer, business, or individual defendant who must compensate the injured individual based on the degree of injuries, income loss, and/or disability that has been described by various medical providers. While many studies have been carried out with subjects who are trying to manipulate diagnostic test results (cite), none have been carried out in a simulated secondary gain environment where subjects are substantially rewarded for their feigned diagnostic testing results (malingering false positives).

Aspects of the invention also include a significant reward for fraudulent behavior which leads to a malingering false positive result. For this to be the case, a significant prize must be provided to the subject in the diagnostic testing group who is the most successful in producing a false positive test. In order for this testing environment to produce the highest likelihood of false positives, the prize must be must be meaningful to the individual. For example, a $100 prize might have little value to someone earning $100,000 a year, but to a blue collar worker who earns $15 an hour, it may be a suitable incentive to want to work hard to manipulate diagnostic tests to show an injury. As a result, while not meant to limit the scope of this invention, the subjects are asked through questionnaire or interview about what would be a meaningful prize. It is believed that a prize equal to several days pay for several hours of testing (or a 400-500% premium) is enough to produce simulated secondary gain behavior.

Aspects of the invention also include that disease and malingering status are blinded to readers of the tests. In other words, the test readers are unaware if a test being read was performed on a normal patient without the disease, a patient known to have the disease, or a subject placed in a simulated secondary gain environment incented to fake a positive test result. In this way, the data generated by the blinded readers can produce both disease and malingering sensitivity and specificity. As an example, if out of 10 tests for the simulated secondary gain group, one were read by the blinded readers as negative, and one was read as positive, the malingering false positive rate for that test is 10%.

Aspects of the invention also include that various diagnostic tests may either be read as positive or negative, but some may also be read as producing an obviously deceptive result. For example, a negative result for a diagnostic test may at times appear quite different than a test being manipulated by an incented subject in a simulated secondary gain trial.

Aspects of the invention also include that a feedback system is utilized to improve test performance to reduce malingering false positive rates. As an example not meant to limit the scope of this invention, a simulated secondary gain trial with 5 subjects evaluating 3 tests may show by blinded reading that one of the tests produces a malingering false positive rate of 30%. This would be considered unacceptably high and would initiate a feedback loop whereby the test results would be analyzed to either change key parts of how the test is read as abnormal or deceptive, change the actual test parameters to further reduce the malingering false positive rate, or drop the test from the diagnostic testing suite as a valid test in a medical-legal environment.

As discussed, aspects of this invention include that the outcome of any simulated secondary gain evaluation of a diagnostic test can only be: A. the test has a acceptably low false positive rate and needs no further versions to reduce that rate B. the test parameters need to be altered or its interpretation altered to further reduce the malingering false positive rate C. the test can't be repaired to produce an acceptable malingering false positive rate and should not be used in a medical legal environment. If the test were deemed repairable, then the new version of either or both of the test reading methodology or the test parameters could be placed back into the same simulated secondary gain environment to test the hypothesis that the repaired version will further reduce the malingering false positive rate. Using this constant feedback loop, it is expected that many tests will evolve to reduce their malingering false positive rate.

Aspects of the invention also include that once a subject is able to simulate a false positive in a simulated secondary gain environment, that same subject and other random subjects should be used to reevaluate the repaired test for its malingering false positive rate. Certainly, if a subject posses certain skills, mental attributes, or physical attributes that makes him or her able to simulate a malingering false positive, then the altered test must be able to become impervious to that same subject (assuming it's malingering false positive rate is still unacceptably high).

Aspects of the invention also include software which will be updated based on the feedback system described above, wherein the test parameters and test reading methodology will evolve to minimize the malingering false positive rate. This software can provide normal, abnormal, or deceptive test readings or can merely transmit or print a report that can be read by a healthcare professional. One implementation of this software could use the World Wide Web or a portal methodology whereby the software is continuously updated based on the data obtained from ongoing simulated secondary gain trials.

### Definitions:

The following definitions are provided to facilitate understanding of certain terms used frequently herein and are not meant to limit the scope of the present disclosure.

“Malingering” refers to the act of consciously deceiving healthcare professionals or others about disease severity or status. In other words, a conscious simulation of an illness (with no organic pathology present) used to avoid an unpleasant situation or for personal gain.

“Secondary Gain” refers to the external gain derived from any illness, such as personal attention and service, monetary gains, disability benefits, and release from unpleasant responsibilities.

“Malingering False Positive” refers to a positive diagnostic test result produced by a subject without the disease who is in a simulated secondary gain environment.

“Simulated Secondary Gain Environment” refers to a testing setting where a group of subjects are given a script where they are to feign an injury or illness and are given a significant prize if they are able to produce a false positive test result.

“Target Test” refers to a diagnostic test that is placed through simulated secondary gain trials to reduce its false positive rates in a medical legal setting.

“Subjects” refers to patients or actors who undergo diagnostic testing during a trial.

“Trial” refers to a group of subjects who undergo diagnostic testing for the purpose of calibrating the test, validating the test, or subjecting the test to a simulated secondary gain model to determine its malingering false positive rate

What is claimed is:

1. A method for minimizing the false positive rate of diagnostic tests in a medical legal environment.

2. The method of claim 1 wherein secondary gain is simulated in a group of subjects without disease by instructing these subjects to feign illness so as to produce a false positive result on a diagnostic test.

3. The method of claim 1 wherein the false positive test data produced by this trial is used to alter test parameters to reduce the prospective false positive rate of future diagnostic tests.

4. The method of claim 1 wherein the false positive test data produced by this trial is used to alter test interpretation to reduce the prospective false positive of future diagnostic tests.

5. The method of claim 1, wherein software or algorithms are used to continuously minimize the false positive rate of diagnostic tests in a medical legal environment.

6. The method of claim 1, wherein a prize or reward is provided to those subjects successfully producing a false positive diagnostic test result.

7. The method of claim 6, wherein the prize or reward provided is deemed by the subjects to be significant enough to produce competitive behavior.

8. The method of claim 7, wherein the prize or reward produces a significant number of false positive diagnostic test results.

9. The method of claim 1 wherein the false positive test data produced by this trial is used to eliminate a certain diagnostic test from being used to settle medical legal disputes over the veracity of injuries or illness.

10. The method of claim 1 wherein the false positive test data produced by this trial is used to improve a certain diagnostic test for the purposes of using the test to settle medical legal disputes over the veracity of injuries or illness.

11. The method of claim 1 wherein the false positive test data produced by this trial is used to eliminate a certain diagnostic test from being used to perform accurate diagnosis in a medial legal setting.

12. The method of claim 1 wherein the false positive test data produced by this trial is used to improve a certain diagnostic test for the purposes of performing accurate diagnosis in a medical legal setting.

13. The method of claim 1 wherein the false positive test data produced by this trial is used to alter test implementation to reduce the prospective false positive of future diagnostic tests.

14. The method of claim 1 wherein the false positive test data produced by this trial is used to alter test machinery to reduce the prospective false positive of future diagnostic tests.

15. The method of claim 1 wherein the false positive test data produced by this trial is used to alter test computer hardware or software to reduce the prospective false positive of future diagnostic tests.
